(19) United States
(12) Patent Application Publication
(10) Pub. No.: US 2024/0324894 A1
(43) Pub. Date: Oct. 3, 2024

(54) BREATH ANALYSIS SYSTEM

(71) Applicant: Breath Diagnostics Inc., Louisville, KY (US)

(72) Inventor: Thu-Ha Thi Duncan, Cleveland, TN (US)

(21) Appl. No.: 18/620,894

(22) Filed: Mar. 28, 2024

Related U.S. Application Data
(60) Provisional application No. 63/455,179, filed on Mar. 28, 2023.
(60) Provisional application No. 63/455,191, filed on Mar. 28, 2023.

(51) Int. Cl. A61B 5/08

(52) U.S. Cl. CPC A61B 5/082

(57) ABSTRACT

A system determines if a breath sample is valid or invalid for subsequent diagnostic analysis. The system includes a breath collection device configured to contain a collected patient breath sample. A cassette has reagents therein and is configured to fluidly couple with the breath collection device and the validity analysis device. A validity analysis device has a fluidic pathway and a controller. The validity analysis device is configured to fluidly couple with the cassette and/or the breath collection device. The analysis device is further configured to evacuate at least a portion of the patient breath sample from the breath collection device, such that the patient breath sample passes through the cassette to define a processed gas sample. A CO2 sensor is configured to determine a CO2 concentration in the processed gas sample. The controller is configured to determine when a patient provided an invalid collected patient breath sample.

### PRIORITY

This patent application claims priority from provisional U.S. patent application No. 63/455,179, filed Mar. 28, 2023, entitled, “BREATH COLLECTION SYSTEM,” and naming Thu-Ha Thi Duncan as inventor, the disclosure of which is incorporated herein, in its entirety, by reference.

This patent application also claims priority from provisional U.S. patent application No. 63/455,191, filed Mar. 28, 2023, entitled, “BREATH ANALYSIS SYSTEM,” and naming Thu-Ha Thi Duncan as inventor, the disclosure of which is incorporated herein, in its entirety, by reference.

### FIELD OF THE INVENTION

Illustrative embodiments of the invention generally relate to detection and monitoring of diseases using breath analysis and, more particularly, illustrative embodiments relate to ensuring accurate sample collection for performing an analysis of the breath.

### BACKGROUND OF THE INVENTION

In the evolving landscape of medical diagnostics, the non-invasive detection and monitoring of diseases through breath analysis has emerged as a groundbreaking approach, leveraging the composition of exhaled breath as a rich source of biomarkers. Breath analysis determines health status by using the presence of volatile organic compounds (VOCs) and other gases that are by-products of metabolic processes within the human body, whose patterns can shift significantly with physiological changes linked to various diseases.

Traditional diagnostic procedures often require invasive, time-consuming, and costly tests that can impose considerable discomfort and stress on patients. In contrast, breath analysis offers a rapid, patient-friendly alternative that holds the potential for early disease detection, real-time monitoring, and personalized treatment strategies, paving the way for advancements in preventative medicine (e.g., early detection) and the management of chronic conditions.

### SUMMARY OF VARIOUS EMBODIMENTS

In accordance with one embodiment of the invention, a system collects samples of patient breaths. The system includes a flexible bag configured to house a gas exhaled by a patient. The system also includes a valve device having a normally closed channel that is transitionable from a closed configuration, wherein gas does not flow through the channel, to an open configuration, wherein gas does flow through the channel. The valve device includes a mouthpiece with a teeth receptacle area configured to receive the patient's front teeth. The channel opens when the patient bites on the teeth receptacle area. A mouth flange is configured to be positioned outside and adjacent to the patient's mouth when the patient bites on the teeth receptacle area. The flange is configured to prevent or reduce airflow towards the patient's mouth.

Some embodiments include a rigid support element positioned at least partly within the flexible bag. The rigid support element may have a threaded post positioned outside of the bag and configured to threadably engage with the valve device. To that end, the valve device may have internal threads configured to engage with the external threads. The valve device may include a valve flange configured to externally support the bag. The system may include an o-ring configured to seal between the post of the support element and the internally threaded valve device.

The mouthpiece may include a second teeth receptacle area, opposite the first teeth receptacle area, configured to receive a user's bottom teeth. A channel may be formed through the valve and the rigid support element. The channel may begin at the proximal end of the mouthpiece. In various embodiments, the channel may have a diameter of between 3 mm and 3.5 mm. In various embodiments, the bag may be formed of material that is permeable to water vapor, but is not permeable to volatile organic compounds.

The system may further include a flexible cap having a luer port configured to seal the gas flow channel of the valve device. The cap may be configured to seal the gas flow channel of the valve device, for example, after the channel is deformed by the patient bite.

Various embodiments may include a microreactor cassette configured to fluidly couple with the bag via the cap. Furthermore, a validity analysis device configured to fluidly couple with the bag and the cassette may be provided.

In accordance with another embodiment, a method analyzes a collected breath sample to determine if the breath is a faulty sample. The method receives a bag containing a collected patient breath sample. A bag is fluidly coupled with a cassette having reagents therein. The cassette is fluidly coupled with an analysis device having a fluidic pathway. The analysis device also has a controller. The method evacuates at least a portion of the patient breath sample from the bag, such that the patient breath sample passes through the cassette and reacts with the reagents therein to define a processed gas sample. At least a portion of the processed gas sample is received within the fluidic pathway of the analysis device. Preferably, the entirety or almost the entirety of the patient breath sample is collected and analyzed. The method analyzes the processed gas sample using a CO2 sensor of the analysis device to detect the concentration of CO2 in the processed gas sample. The method determines when a patient provides a faulty collected patient breath sample by comparing the CO2 concentration of the processed gas sample with a known faulty CO2 concentration.

In various embodiments, the method provides an alert that the collected patient breath sample was faulty. The analysis device is configured to provide the alert within 1-minute of the processed gas sample being received within the fluidic pathway of the analysis device. However, in some embodiments, the alert may be provided within 5-minutes, 10-minutes, 15-minutes, or other timing as desired. Generally, the alert is provided quickly to allow for rapid retesting in case of faulty breath. The processed gas sample may be determined to be faulty if it contains less than about 2.5% CO2.

In accordance with yet another embodiment, a method analyzes a collected breath to determine if the breath is faulty for diagnostic breath analysis. The method receives a collected patient breath sample in a breath collection device. The breath collection device is fluidly coupled with an analysis device having a fluidic pathway and a controller. At least a portion of the patient breath sample is evacuated from the breath collection device. The at least a portion of the collected patient breath is received within the fluidic pathway of the analysis device. The at least a portion of the collected patient breath sample is analyzed using a CO2 sensor of the analysis device to detect the concentration of CO2. The method determines when the patient provides a faulty collected patient breath sample by comparing the CO2 concentration of the at least a portion of the collected patient breath sample with a known faulty CO2 concentration.

In various embodiments, the breath collection device comprises a bag having a normally closed valve. The breath collection device may include a bag having a normally closed valve with a teeth receiving area. The valve may be configured to open when a patient bites on the teeth receiving area. Some embodiments control a pump to control a fluid flow rate through the fluidic pathway of the analysis device and/or to extract gas from the breath collection device.

In response to an alert of a faulty collected breath sample, the method may receive a second collected patient breath sample. The breath collection device may again be fluidly coupled with an analysis device. At least a portion of the second patient breath sample from the breath collection device is evacuated, such that at least a portion of the second collected patient breath is received within the fluidic pathway of the analysis device. The at least a portion of the second collected patient breath sample is analyzed using a CO2 sensor of the analysis device to detect the concentration of CO2 in the at least a portion of the second collected patient breath. The method determines whether the patient provided a faulty second collected patient breath sample by comparing the CO2 concentration of the at least a portion of the second collected patient breath sample with a known faulty CO2 concentration.

The steps of collecting subsequent patient breath samples, evacuating at least a portion of the collected subsequent patient breath samples, analyzed the at least a portion of the collected subsequent patient breath samples, and determining when a patient provided a faulty subsequent patient breath samples may be repeated until a valid sample is obtained. Some embodiments may identify whether the faulty samples are caused by lack of alveolar breath or a leak in the channel.

In accordance with yet another embodiment, a method determines if the breath sample is valid for diagnostic analysis. The method receives a bag having a collected patient breath sample therein. The bag is fluidly coupled with an analysis device having a fluidic pathway and a controller. At least a portion of the patient breath sample is evacuated from the bag, such that at least a portion of the collected patient breath sample is received within the fluidic pathway of the analysis device. The method determines when a patient provided a valid collected patient breath sample by comparing the CO2 concentration of the at least a portion of the collected patient breath with a known valid CO2 concentration.

Various embodiments may provide an alert that the collected patient breath was valid or not valid. Various embodiments to provide the alert within 1-minute of the gas sample being received within the fluidic pathway of the analysis device.

In accordance with yet another embodiment, a system determines if a breath sample is valid or invalid for subsequent diagnostic analysis. The system includes a breath collection device configured to contain a collected patient breath sample and to fluidly couple with a cassette and/or a validity analysis device. The cassette has reagents therein and is configured to fluidly couple with the breath collection device and the validity analysis device. A validity analysis device has a fluidic pathway and a controller. The validity analysis device is configured to fluidly couple with the cassette and/or the breath collection device. The analysis device is further configured to evacuate at least a portion of the patient breath sample from the breath collection device, such that the patient breath sample passes through the cassette to define a processed gas sample. A CO2 sensor is configured to determine a CO2 concentration in the processed gas sample. The controller is configured to determine when a patient provided an invalid collected patient breath sample.

In various embodiments, the controller determination may be a function of comparing the CO2 concentration of the processed gas sample with a known invalid CO2 concentration. The controller determination may also be a function of volume of the processed gas sample. The validity analysis device may include a pump.

In accordance with a further embodiment, a system analyzes a collected breath sample to determine if the breath sample is valid or invalid for subsequent diagnostic analysis. The system includes a validity analysis device having fluidic pathway and a controller. The validity analysis device is configured to fluidly couple with a cassette and/or a breath collection device. The analysis device is further configured to evacuate at least a portion of the patient breath sample from the breath collection device. A controller is configured to receive a CO2 concentration in the collected breath sample from a CO2 sensor. The controller determines when a patient provided a valid or invalid collected breath sample. The controller is further configured to provide an alert when the collected breath sample is valid or invalid.

The system may include a breath collection device configured to contain the collected patient breath sample and to fluidly couple with a cassette and/or a validity analysis device. A microreactor cassette may have reagents therein. The cassette may be configured to fluidly couple with the breath collection device and the validity analysis device.

In various embodiments, the system may include a flexible bag configured to house a gas exhaled by a patient. The system may also include a valve device having a normally closed channel that is transitionable from a closed configuration, wherein gas does not flow through the channel, to an open configuration, wherein gas does flow through the channel. The valve device may include a mouthpiece with a teeth receptacle area configured to receive the patient's front teeth. The channel may open when the patient bites on the teeth receptacle area. A mouth flange may be configured to be positioned outside and adjacent to the patient's mouth when the patient bites on the teeth receptacle area. The flange may be configured to prevent or reduce airflow towards the patient's mouth.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIGS. 1A-1D schematically show a patient providing a breath sample for analysis in accordance with illustrative embodiments.

FIG. 2 shows a process of determining whether a patient sample is valid in accordance with illustrative embodiments.

FIGS. 3A-3F schematically show a breath collection device in accordance with illustrative embodiments.

FIGS. 4A-4B schematically show the breath collection device of FIGS. 3A-3F fluidly coupled with the analysis station in accordance with illustrative embodiments.

FIG. 5 schematically shows the analysis station simultaneously coupled with a plurality of bags for simultaneous analysis.

FIG. 6 schematically shows details of the fluid system controller 120 in accordance with illustrative embodiments of the invention.

### DESCRIPTION OF ILLUSTRATIVE EMBODIMENTS

In illustrative embodiments, a breath analysis system (also referred to as an evacuation station) receives a sample of exhaled breath collected from a patient. The sample is collected in a breath collection apparatus (e.g., including an inflatable bag), and then extracted by the system, which may pass the collected fluid sample (e.g., gas) through a microreactor having reagents therein. The breath analysis system rapidly determines whether the collected breath sample is a valid sample from which medical diagnostic information can be reliably determined.

Furthermore, illustrative embodiments provide a breath collection device configured to increase the percentage of valid collected breath samples. The breath collection system includes a bag having a mouthpiece with a valve. The valve has a channel through which patient exhaled breaths pass to reach the interior of the bag. The mouthpiece has a teeth receptacle area configured to receive a user's teeth (e.g., the user's front teeth), such that the valve is configured to open the channel and allow exhalation when the user bites down on the tooth receptacle area. The mouthpiece includes a flange positioned outside and adjacent to the patient's mouth as the user bites on the tooth receptacle area (e.g., when the patient is preparing to provide a breath to the breath collection system). The flange is configured to prevent or reduce external airflow to/from the user's mouth (i.e., other than through the channel of the valve) thereby increasing validly collected breath samples. Some embodiments include a rigid support element (such as a ring) inside the bag to prevent the bag from collapsing in an undesirable manner and preventing the complete removal of collected air from inside the bag. Details of illustrative embodiments are discussed below.

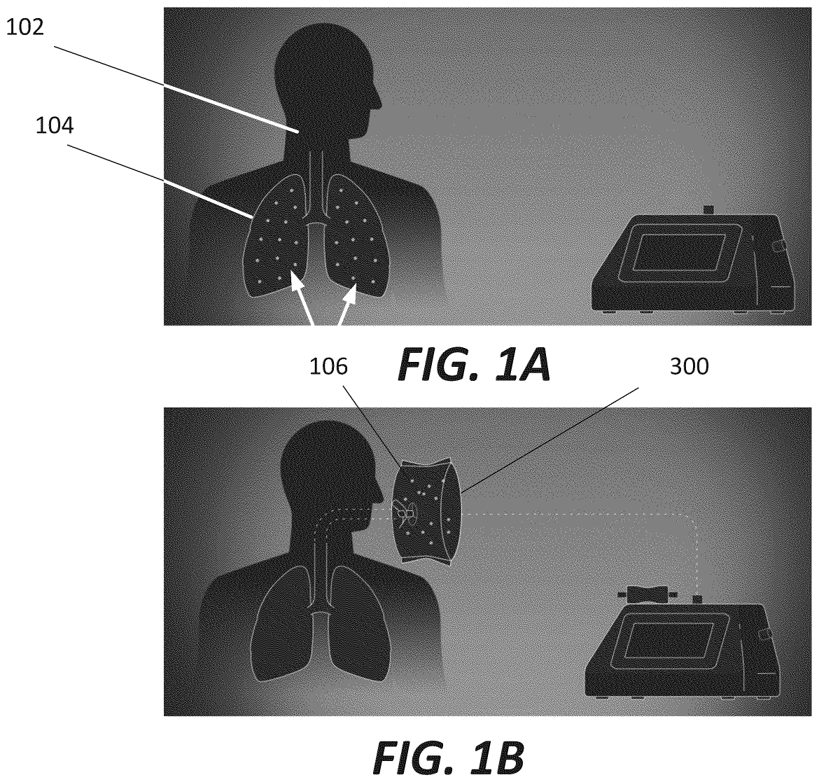

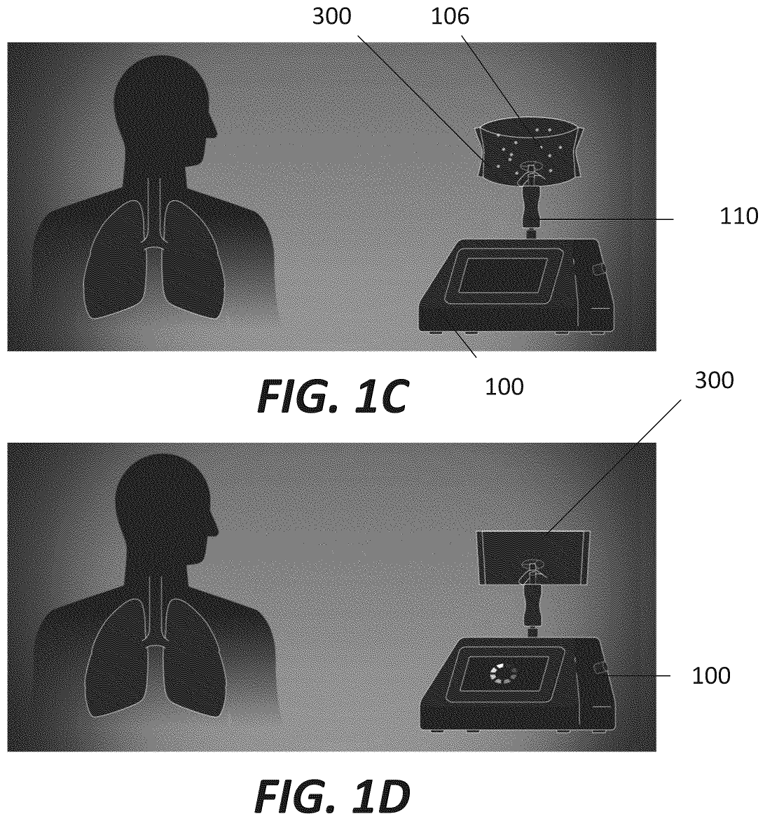

FIGS. 1A-1D schematically show a patient 102 providing a breath sample for analysis in accordance with illustrative embodiments. In particular, FIGS. 1A and 1B show the patient 102 providing a sample from the lungs 104 to the breath collection system 300. FIGS. 1C-1D show the collected sample being coupled with and processed by a breath analysis system.

FIG. 1A shows the patient 102 having a volume of gas within their lungs 104. The gas volume contains volatile organic compounds (VOCs 106) among other things. The patient 102 is provided with a sample collection device 300 (also referred to as a breath collection device 300), such as a collection bag, tube, or directly analyzed by the diagnostic instrument. The individual then exhales into the collection device 300, thereby transferring the VOCs 106 and other components of the breath into the collected device. If the patient 102 does not provide the sample correctly, parts of the VOCs 106 or other analyzed components may not be collected in the collection device 300, rendering the sample invalid.

FIG. 1B shows the gas from the patient 102 lungs 104 transferred into the collection device 300. Various embodiments collect and analyze alveolar breath. Alveolar breath refers to the air that is exhaled from the deepest part of the lungs 104, specifically from the alveoli, which are tiny air sacs where the exchange of oxygen and carbon dioxide with the blood takes place. This part of the exhaled breath is of particular interest in breath analysis and medical diagnostics because it is rich VOCs 106 that can provide valuable insights into the body's metabolic processes. Alveolar breath is considered to be the most representative of the body's internal chemical state, making it a prime target for analyzing and identifying biomarkers related to various diseases and conditions. The composition of alveolar breath can reveal information about the lungs 104 and other parts of the body, offering a non-invasive means to diagnose, monitor, and study health and disease.

FIG. 1C shows the collection device 300 fluidly coupled with an analysis system. Optionally, some embodiments may include a microreactor that is fluidly coupled between the collection device 300 and the analysis system 100. The microreactor includes a reagent that traps the VOCs 106, stabilizing them. The microreactor may then be separated from the remainder of the system and shipped to a lab for analysis, for example, when the breath analysis system 100 rapidly determines that the collected breath sample is a valid sample from which medical diagnostic information can be reliably determined.

The gas from the device, including the VOCs 106, is evacuated from the breath collection system 300 through the microreactor and into the analysis system 100, where the gas is analyzed for validity. The gas may pass through a fluidic cassette or microreactor before being received by the analysis system 100. FIG. 1D shows the analysis system 100 processing the collected breath. As described further below, the analysis system 100 may then determine and report whether the collected sample was validly collected. In some embodiments, the analysis system 100 may also perform further analysis and/or diagnosis on the collected sample (e.g., instead of shipping out the microreactor to an external lab).

If the collected breath was validly collected, the collected breath sample may be used to detect and analyze the volatile organic compounds and other gases in the collected breath, utilizing specific biomarkers to diagnose and monitor various diseases non-invasively. In various embodiments, specific VOCs 106 or patterns of VOCs 106 serve as biomarkers for particular diseases. The presence, absence, or concentration of these biomarkers can indicate the presence of disease, including, for example:
Respiratory Diseases: Conditions such as asthma, chronic obstructive pulmonary disease (COPD), and lung cancer can alter the composition of exhaled breath.
Metabolic Disorders: Diabetes can be detected through the analysis of acetone levels in the breath, which are elevated when the body burns fat for energy instead of glucose.
Infections: Certain bacteria and viruses can produce specific VOCs 106. For example, Helicobacter pylori, a bacterium associated with stomach ulcers and cancer, can be detected through breath analysis.

It should be apparent that the rapid collection of valid breath samples for breath analysis provide a number of advantages, including:
Non-invasive: Collecting breath samples is easy, painless, and can be performed repeatedly without risk to the patient 102.
Rapid collection & recollection: provide results more quickly than traditional laboratory tests, with confidence that the sample is valid. If the valid is improperly collected, patient 102s do not have to wait weeks to determine that the sample is improperly collected and then to recollect.
Potential for Early Detection: Breath analysis can potentially detect diseases at an early stage, improving treatment outcomes.

As mentioned above, illustrative embodiments advantageously provide an indication of when a valid or invalid breath sample has been collected. Proper collection of a breath sample advantageously improves the accuracy and reliability of breath diagnostic analysis, as it ensures the consistent measurement of volatile organic compounds (VOCs 106) and other biomarkers related to diseases. Incorrect or inconsistent sampling can introduce variability and contamination, leading to false positives or negatives, and potentially compromising the diagnostic process. By providing an improved sample collection device 300, illustrative embodiments minimize external influences and physiological variations, thereby achieving reliable diagnostic outcomes. Additionally, early detection of an invalid sample facilitates early disease detection and enables effective monitoring and management of health conditions using breath analysis.

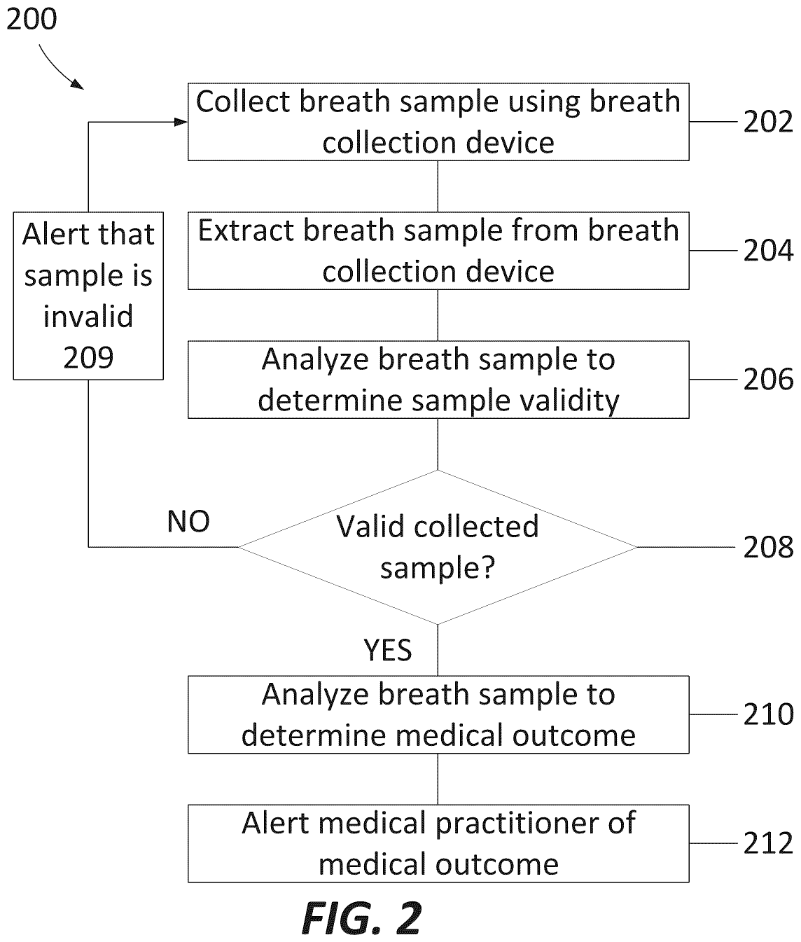

FIG. 2 shows a process 200 of determining whether a patient 102 sample is valid in accordance with illustrative embodiments. It should be noted that this method is substantially simplified from a longer process that may normally be used. Accordingly, the method shown in FIG. 2 may have many other steps that those skilled in the art likely would use. In addition, some of the steps may be performed in a different order than that shown, or at the same time. Furthermore, some of these steps may be optional in some embodiments. Accordingly, the process 200 is merely exemplary of one process in accordance with illustrative embodiments of the invention. Those skilled in the art therefore can modify the process as appropriate.

The process 200 begins at step 202, which collects a breath sample from the patient 102 using the breath collection device 300. Preferably, the sample includes an alveolar breath sample. In various embodiments, the patient 102 is provided with the breath collection device 300 and instructed to, for example:
1. Put a mouthpiece of the breath collection device 300 into their mouth and bite on to the teeth receptacle of the mouthpiece;
2. Close their lips around the mouthpiece such that the flange is positioned around their mouth;
3. While breathing normally, begin blowing into the bag as much as they can through their mouth, and do not breath through the nose.
4. Remove the mouthpiece and hand the breath collection device 300 to the staff to cap,

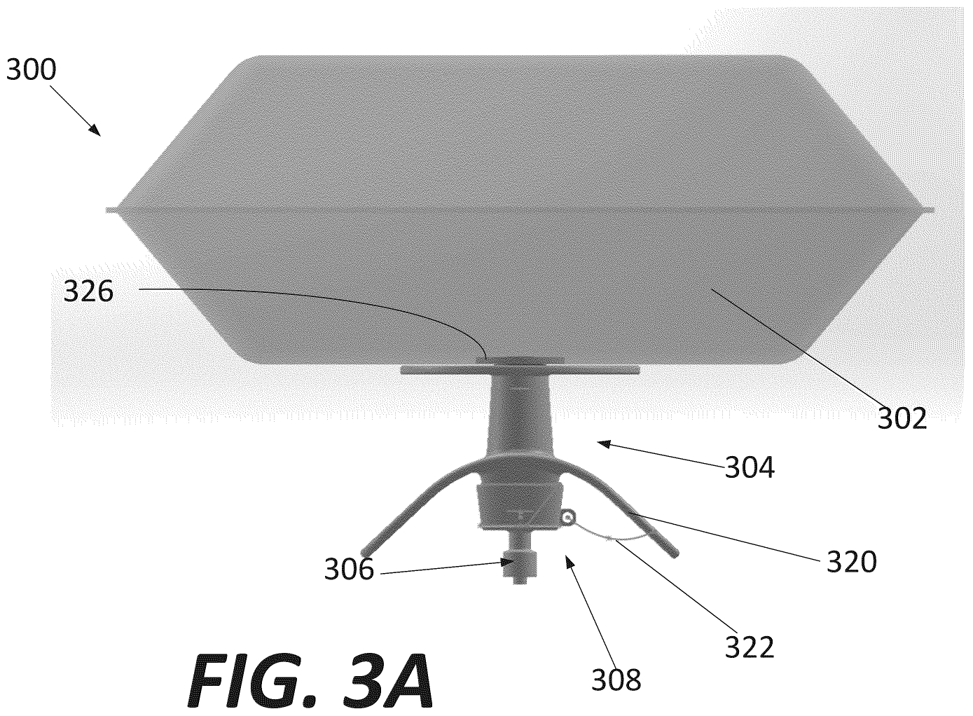

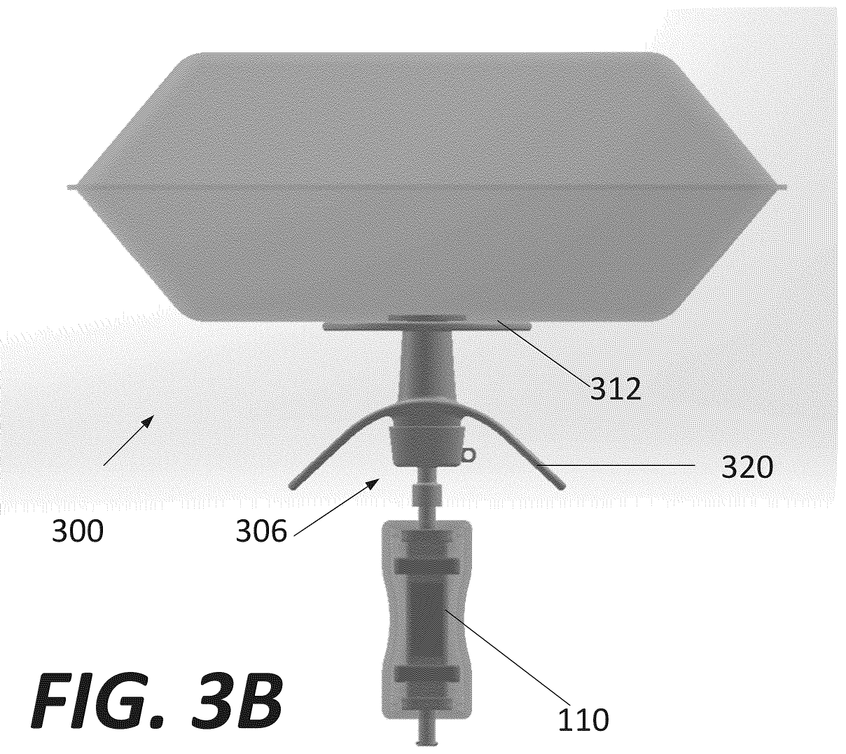

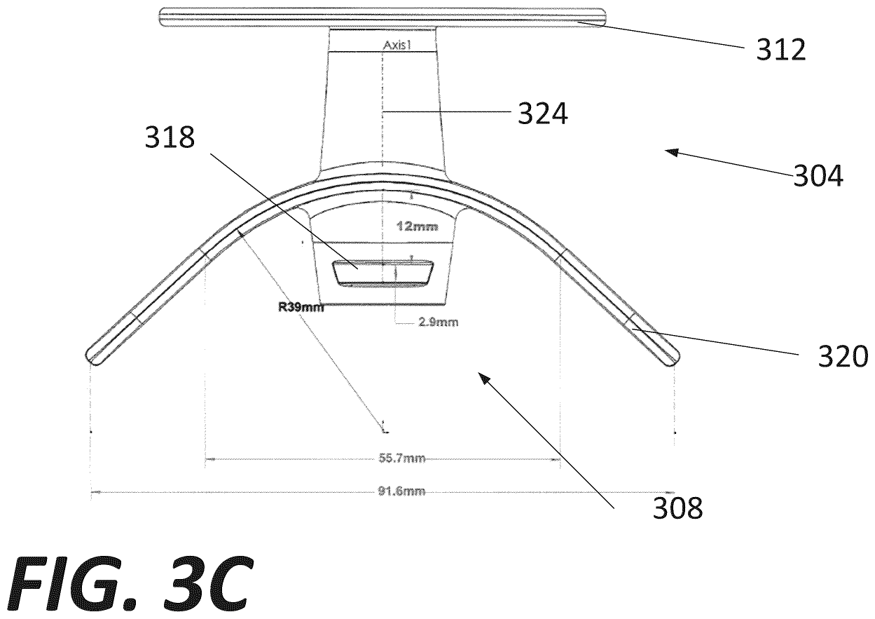

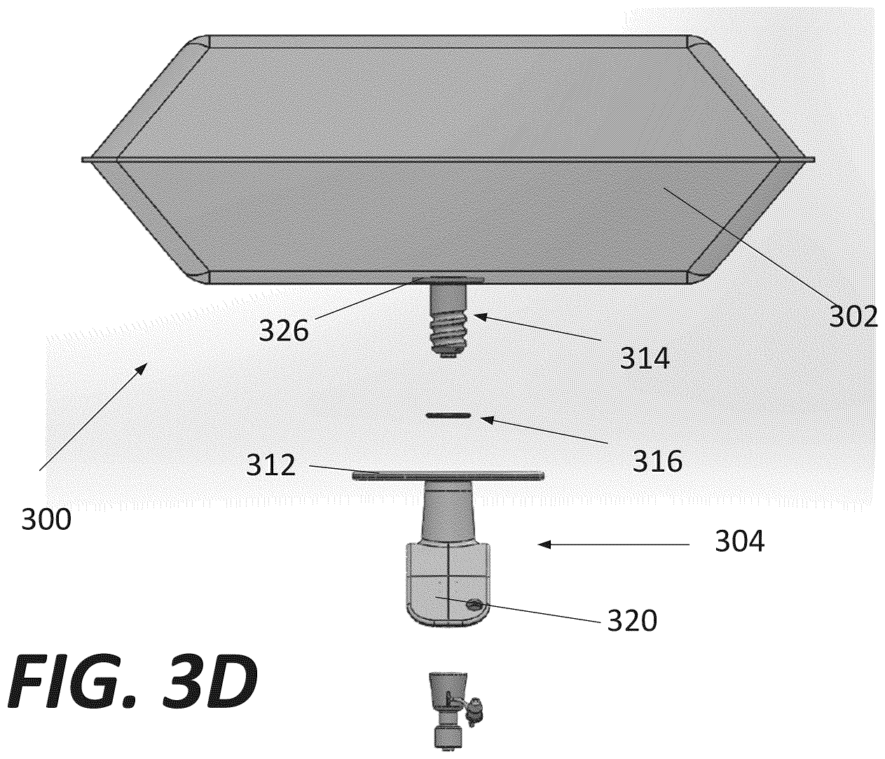

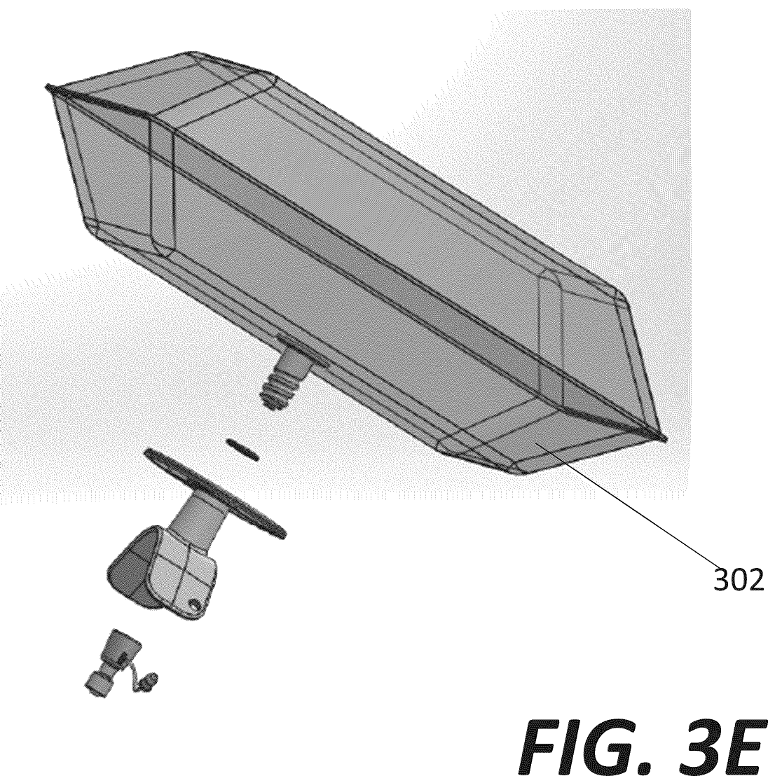

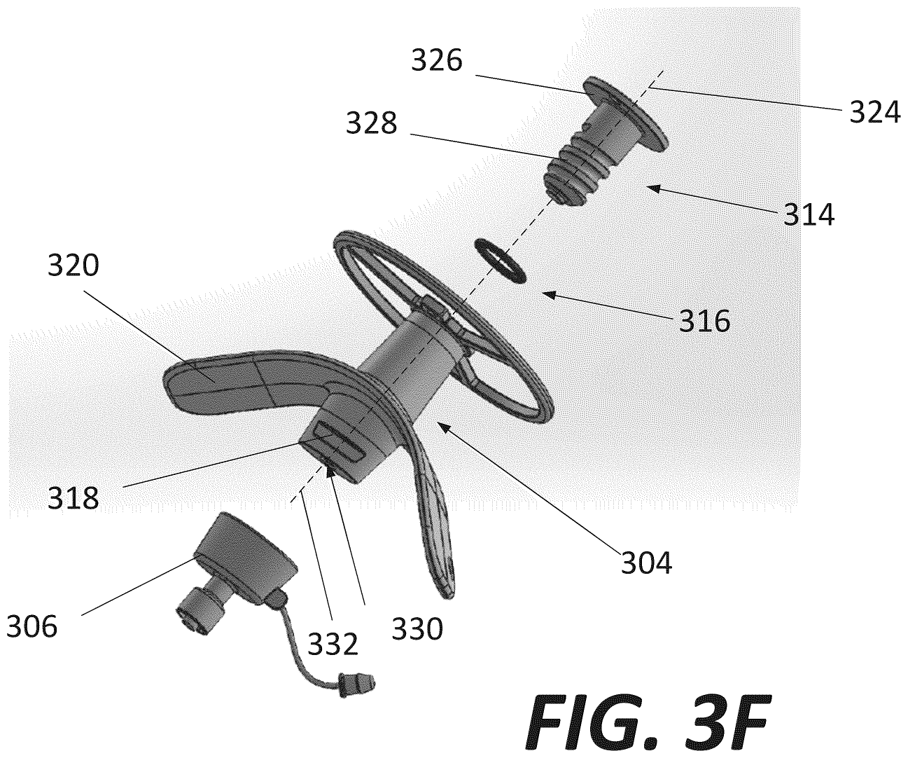

FIGS. 3A-3F schematically show a breath collection device 300 in accordance with illustrative embodiments. In illustrative embodiments, the breath collection device 300 advantageously improves the collection of alveolar patient 102 breath for the purposes of analyzing the VOCs 106. Accordingly, the breath collection device 300 advantageously results in a higher number of valid samples for alveolar breath analysis.

FIG. 3A shows the collection device 300 having a container, such as bag 302, for housing the collected breath in accordance with illustrative embodiments. The bag 302 may be formed of a thin film that is impermeable to VOCs 106, but permeable to water vapor. For example, illustrative embodiments form the bag from Tedlar™ Film (Dupont®). As another example, the bag 302 may be formed from polyvinyl fluoride coated with a material that keeps the VOCs 106 from permeating through the bag 302, e.g., polyethylene film, polyvinylidene fluoride (PVDF) film such as KYNAR™ film available from Arkema, polyvinyl fluoride (PVF) film such as TEDLAR™ film available from Dupont, without limitation. Some other materials may undesirably capture the patient breath sample and allow the VOCs 106 escape. Illustrative embodiments preferably remove some or all of the humidity from the bag 302 because the VOCs 106 tend to be dissolvable in liquid. Some embodiments analyze the gas, as opposed to any liquid inside the bag 302, and therefore, it is undesirable to have VOCs 106 dissolve in the liquid.

In FIG. 3A, the breath collection apparatus 300 includes a valve 304 through which patient breath passes to enter into the bag 302. In various embodiments, the valve 304 is preferably normally closed, such that it opens under certain conditions (e.g., when a patient 102 bites down on the valve) and otherwise remains closed. In various embodiments, the valve 304 may deform after patient 102 biting, and therefore, it may be desirable to include a cap 306 that prevents unintentional escape of any collected fluid within the bag 302. In particular, the cap 306 may be configured to fit over a mouthpiece 308. In some embodiments, the mouthpiece 308 may be integrally formed with the valve 304, or otherwise fluidly coupled with the valve.

FIG. 3B schematically shows the breath collection device 300 coupled with a microreactor cassette 110 via the cap 306. The cap 306 may include a luer interface for coupling with the cassette 110. The cassette 110 houses the microreactor. The microreactor is used to provide advantageous reactions for an associated diagnostic analysis device. The collected sample reacts with chemicals inside of the cassette 110 as it is drawn out of the bag 302 towards the gas analysis system 100.

FIG. 3D schematically shows an exploded view of the device 300 of FIG. 3A rotated 90 degrees. FIG. 3E schematically shows another view of the device 300 of FIG. 3D. FIG. 3F schematically shows an exploded detailed view of the device 300 without the bag 302.

In various embodiments, a valve flange 312 interfaces with a threaded post 314 (also referred to as a support element 314) that passes through an opening of the bag 302. The bag 302 is coupled with the threaded post 314, which is at least partially internally to the bag. To that end, the post 314 includes a support flange 326 for sealing coupling the post 314 to the bag 302. The post 314 may also include external threads 328 configured to interface with internal threads in the valve device 304 (not shown in the figures).

In various embodiments, the valve device 304 may be threaded over the post 314 (e.g., the internal threads of the valve mate with the external threads of the post 314). An o-ring 316 may be nested inside of that valve device 304. As the post 314 and the valve device 304 are threaded together, the support flange 326 is compressed against the o-ring 316 and seals the channel 330, reducing or preventing unintended leaks. In various embodiments, the valve flange 312 helps keep the bag 302 sealed, and furthermore acts as a physical support to maintain a desirable structural integrity of the bag 302.

FIG. 3C schematically shows details of the valve 304 in accordance with illustrative embodiments. In various embodiments, the cap 306 is removed from the mouthpiece 308 of the valve 304 when the patient 102 uses the device 300 to collect their breaths. The dimensions shown in FIG. 3C are for example purposes only, and are not intended to limit various embodiments of the invention. For example, in some embodiments, the dimensions shown in FIG. 3C may have +−20% tolerance.

Various embodiments include the channel 330 in the valve 304. The valve channel 330 preferably does not leak under low pressure/vacuum. In various embodiments, the channel 330 may be formed at least in part by an externally threaded post 314 that threadingly engages with an internally threaded portion of the valve 304. Thus, the channel may run from the valve 304 (e.g., from the mouthpiece 308), through the post 314 into the bag 302. The post 314 advantageously provides structural integrity and/or constant pressure to the valve 304 when coupled together. An o-ring 316 may be seated into a groove (not shown, but inside of the valve device 304) in the valve 304 to help seal the channel 330. Furthermore, the threaded coupling between the post 314 and the valve 304 advantageously allows for disassembly and reassembly if a leak is detected.

Various embodiments include a mouthpiece 308 that is configured to limit the patient 102 from undesirably taking additional breath during exhalation. The mouthpiece 308 includes a teeth receptacle area 318 (e.g., a groove) configured to receive the front teeth when the patient 102 bites down on the mouthpiece 308. The inventor found that biting down on the mouthpiece 308, particularly on the teeth receptacle area 318, inhibits or prevents the patient 102 from undesirably taking an inhalation through the mouth, which may cause an invalid analyzed breath. Although preferably having a receptacle area for a plurality of teeth 318, the teeth receptacle area 318 may also be configured to receive a single tooth. The mouthpiece flange 320 has a curvature ergonomically designed to cup the face to cover the mouth after the lips are closed. The mouthpiece flange 320 therefore helps to reduce or prevent airflow to the patient's 102 mouth, thereby reducing or preventing unwanted breaths during the sample collection process.

When providing a breath sample, patient 102 naturally tend to want to take a breath instead of emptying their lungs 104. The inventor determined through testing that having the physical mouth flange 320 reduces the likelihood that the patient 102 collects an invalid alveolar breath sample (e.g., by breathing mid-collection instead of emptying their lungs 104). Furthermore, the inventor has determined that the act of biting down on the mouthpiece 308 advantageously reduces the likelihood that the patient 102 accidentally opens their mouth during sample collection. The mouthpiece flange 320 assists in accurate performance of the protocol for collecting breath (e.g., particularly alveolar breath). In various embodiments, the protocol for collecting a sample generally involves the steps of providing the breath collection device 300 to the patient 102, instructing the patient 102 to bite on the teeth receptacle area 318 and closing their lips around the mouthpiece 308, instructing the patient 102 not to breathe in, and instructing the patient 102 to start breathing out until they can't exhale anymore or they have exhaled all of their breath. This typically results in a collected gas volume of between about 500 mL and about 3 L).

Furthermore, the length of a distance between the bite groove and curvature fits most people comfortably (e.g., about 1 cm to about 1.5 cm from the groove to the mouth flange, preferably about 12 mm).

In various embodiments, the gas flow channel 330 in the mouthpiece 308 is appropriately sized to allow free exhalation. Current gas sampling bags ports are small and limit a person's ability to exhale (especially those with diminished lung function). Prior art gas collection valve devices have tiny flow channels, and it is difficult to get a full breath out by blowing out. The patient 102 undesirably ends up expending a lot of energy without providing a full breath. In contrast, illustrative embodiments include a larger diameter gas flow channel 330 for human use (e.g., for detection of VOCs 106 in human breath in the PPB order of magnitude). In various embodiments, the inventor determined that an advantageous diameter for the channel 330 for collection of human breath gas samples is about 3 mm-to about 3.5 mm based on a balance of volume to pass from human into bag without feeling a restriction and a size expected to accommodate all adults, although other diameters may be practicable in alternative embodiments. In some cases, channel size may be determined or limited by other factors such as the threaded post 314 or by the valve itself.

As described previously, various embodiments include the flexible sealing cap 306. For example, the cap 306 may include a luer lock at the tip and/or a luer taper design adjacent to the mouthpiece. The flexible sealing cap 306 may use a Luer Lock design, and/or may be formed from a pliable/flexible material (such as TPU, silicone or rubber). The flexible cap 306 allows the medical practitioner to press the flexible cap 306 into the taper until it is sealed. If the patient 102 were to bite too hard onto the mouthpiece 308, or have a lot of spit when they are providing the exhaled breath, the flexibility and softness of the cap 306 allows the cap 306 to deform to seal the gas flow channel 330 and make up for those defects (i.e., prevent the breath from leaking out of the bag 302).

Generally, because the bag 302 is made up of a flexible material, it can expand to accommodate the volume of breath provided and in some cases can make up for things that are hard to predict, e.g., moisture content in breaths, which can vary from person to person. It should be noted that the valve material generally is rigid, e.g., in order to prevent the patient 102 from restricting the flow through the channel 330 such as by biting too hard and to allow a means for attachment to the evacuation station that is rigid and will seal every time. In various embodiments, the valve may be formed of a non-flexible material to reduce the likelihood of the patient 102 restricting the flow by biting too hard and to allow a means for attachment to the evacuation station 100 that is rigid and reliably seals.

In various embodiments, the patient 102 receives the bag 302 having the cap 306. The patient 102 removes the cap 306 and provides one or more breaths without the cap 306 blocking the gaseous flow channel 330 through the valve 304 and through the threaded post 314. Then, the clinician uses the cap 306 to plug the gaseous flow channel 330 (even if it is deformed after use). In various embodiments, the mouthpiece may be formed from PTFE. If the channel 330 is deformed because of a hard bite, the cap 306 advantageously deforms to cover the channel 330. Some embodiments may include a rigid non-deformable cap 306, but undesirably, it the rigid cap 306 may leak if the gaseous flow channel 330 doesn't remain the intended shape. FIG. 3F schematically shows an exploded view including a channel axis 332 (e.g., longitudinal axis) formed through an undeformed channel 330.

In various embodiments, the material of the cap 306 may have a hardness that is between Shore A 60-100. In various embodiments, the self-sealing cap is preferably easy to couple to the mouthpiece and tethered thereto via a tether 322. The self-sealing cap may include a Luer Lock and Luer Slip Design interface. A number of tools (syringes and adapters) may be used with the self-sealing cap. In various embodiments, the cap interface may be configured to interface with an analysis device configured to analyze the patient 102 exhaled air captured in the balloon (e.g., the device may have its own counterpart luer interface).

Although step 202 refers to collecting the breath sample using the breath collection device 300 described herein, it should be understood that any breath collection device 300 may be used with various embodiments.

Returning to FIG. 2, after the breath is collected, the process proceeds to step 204, which extracts/evacuates the breath from the breath collection device 300. To that end (and as shown in FIG. 1C) the collection device 300 may be fluidly coupled with the validity analysis system 100. The analysis system 100 may include an internal pump or pneumatic driver that, for example, generates a negative pressure to extract the collected breath sample from the collection device 300. Accordingly, the analysis system 100 may also act as an evacuation system. Additionally, or alternatively, a positive pressure may be applied to the bag 302 to cause the collected gas to be expelled.

As shown in FIG. 1C, various embodiments may advantageously fluidly couple a microreactor 110 between the bag 302 and the analysis system 100. Accordingly, when the breath sample and the collected VOCs 106 flow towards the analysis system 100, the VOCs 106 may be captured in by the microreactor 110. The microreactor 110 may then be analyzed for diagnostic purposes (e.g., after the analysis system 100 determines that the sample is valid). The remaining gas may be referred to as a processed gas sample because of the removal or capture of certain components from the gas by the microreactor. However, the processed gas sample does not require removal or capture of certain components by the microreactor 110. Thus, the term is merely used to describe that the gas has passed through the microreactor 110 on its way to the analysis station 100. In fact, the processed gas sample may be identical to the collected gas sample.

Although illustrative embodiments refer to analysis of VOCs 106 with the patient breath, it should be understood that various embodiments are not limited to VOCs 106. VOCs 106 are provided as an example of a current metabolic by-product that may be used for medical diagnostic purposes. However, it is envisioned that various embodiments may analyze other components of the collected breath sample. Therefore, it should be understood that any discussion of collection or analysis of VOCs 106 also applies to other components of the collected sample.

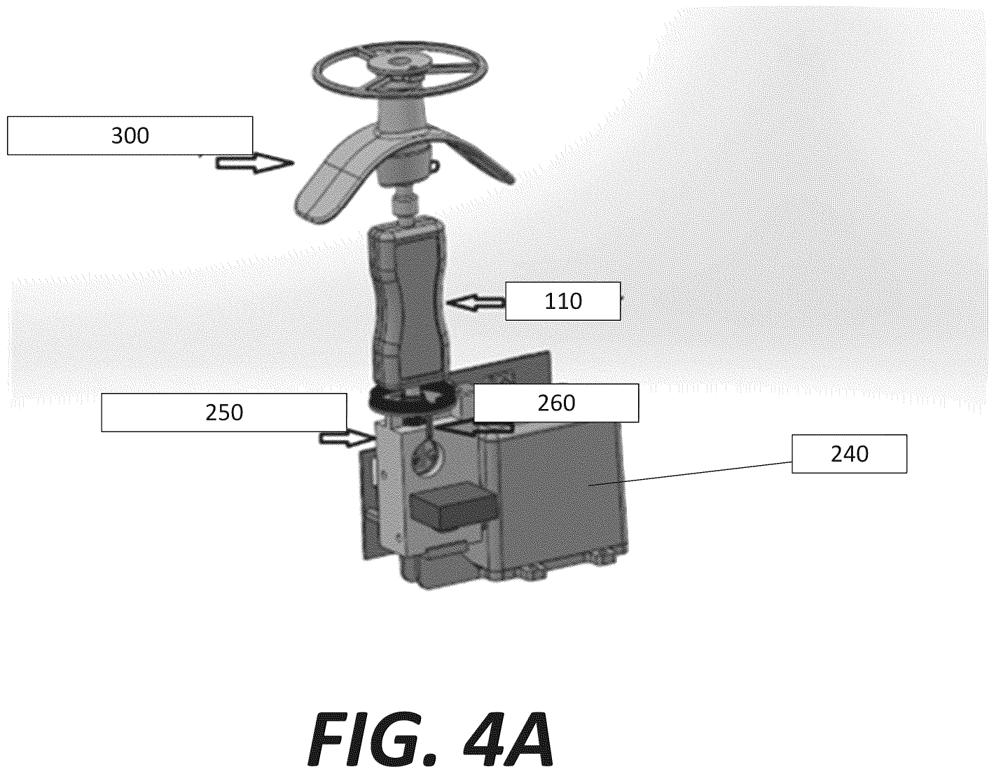

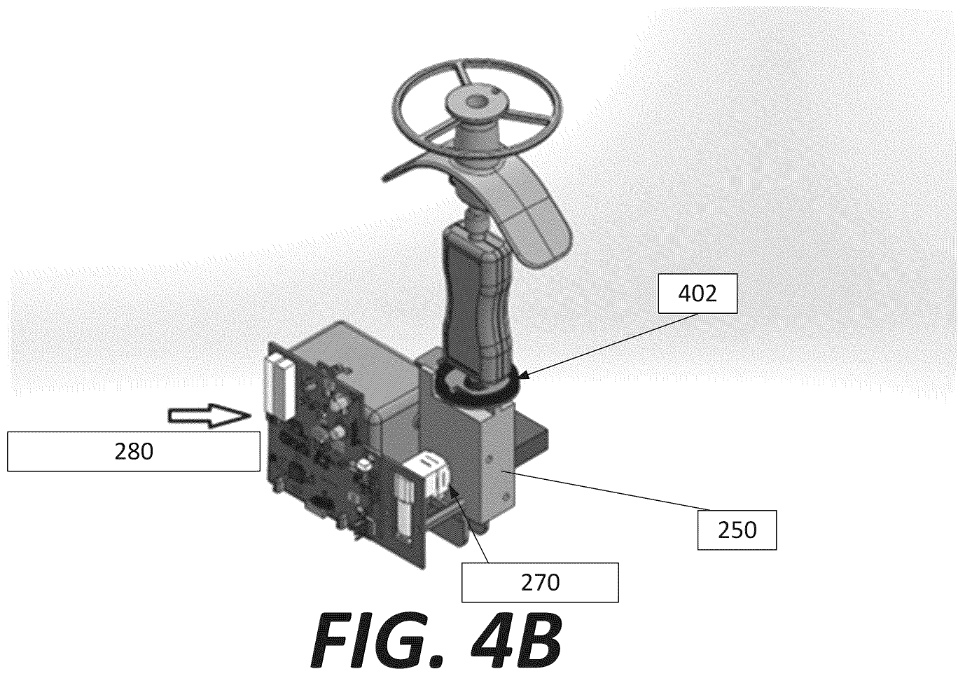

The process then proceeds to step 206, which analyzes the breath sample to determine breath sample validity. FIGS. 4A-4B schematically show the breath collection device 300 fluidly coupled with the microreactor 110 and the analysis station 100 in accordance with illustrative embodiments. Specifically, the valve 304 and the cap 306 of the breath collection device 300 are shown, but it should be understood that the bag 302 is present. The bag 302 is merely omitted for the sake of discussion in FIGS. 4A-4B. Furthermore, a housing of the analysis system 100 is omitted to better show internal components of the analysis system 100.

To analyze the breath sample, the analysis system 100 is configured to control the flow of the breath sample through a fluid path that includes the breath collection device 300, the microreactor 110 (if present), and one or more sensors, including a CO2 sensor 260. The breath is evacuated from the collection device 300 (e.g., using an internal pump of the system 100), passed through the microreactor 110 for VOC collection, and then enters the system 100 for validity analysis.

In various embodiments, the CO2 sensor 260 generates CO2 data that is used by a controller to determine the validity of the breath sample. Thus, illustrative embodiments determine a CO2 content of the breath sample using a CO2 sensor 260 and use the CO2 sensor data to determine if the breath sample is valid for medical diagnostics (e.g., by analysis the VOCs 106 of the breath sample, such as those captures by the microreactor 110).

Preferably, the fluid path within the analysis system 100 is formed using microfluidic channels or pathway 250 (not to be confused with the channel 330 in the collection device, although the pathway 250 and the channel 330 become fluidly coupled) that provide desirable fluid mechanics within the station for the validity analysis. Advantageously, the microfluidics chamber 250 allows for elimination of tubing to reduce failure points. Additionally, the analysis system 100 may have multiple input ports for analysis of multiple breath samples simultaneously. The chamber 250 is configured to provide a closed system for each of the microfluidic so that there is no cross-contamination of the breath samples.

In various embodiments, the aforementioned sensors are positioned within the housing of the analysis system 100. In various embodiments, the sensors 100 may also include a flow sensor 270, among other things.

The inventor determined that real time analysis of the CO2 composition of the collected breath sample reliably allows for determination of whether the breath is valid for diagnostic analysis (e.g., VOC analysis). Accordingly, in various embodiments, the CO2 content of the collected breath sample may be used as a quality metric to determine validity of the breath sample.

In various embodiments, to perform the analysis of step 206, some or all of the gas in the bag may be evacuated out of the bag and into the analyzer for 1 minute, during which, the analyzer analyzes the CO2 content of that breath sample. When the breath sample does not meet a minimum level of CO2 concentration, then it is rejected as an invalid/faulty sample breath.

For example, the evacuation system may detect the concentration of CO2 in the exhalation sample, and determine whether the sample indicates that a user performed the exhalation collection incorrectly (e.g., inhaled during the test, which is not appropriate in some embodiments).

Based on testing, the inventor determined that a faulty breath has a CO2 concentration of less than 2.5%. Thus, in various embodiments, if the analyzer detects a CO2 concentration of less than 2.5% an error alert is provided indicating that there is insufficient alveolar breath. This determination may be made within 1 minute, within 5 minutes, and preferably within 15 minutes. This determination may be made prior to VOC analysis. This data was collected particularly for non-large inhale prior to sample collection, as described in the collection process above, wherein the total breath volume is between about 350 mL and about 2,500 mL of breath.

Some embodiments may operate with a breath collection process that allows a patient 102 to have a large inhale (tidal breath) prior to providing the breath sample. While this is not the preferred method of sample collection, due to the larger volume of gas that requires increased processing time, it is not considered a commercially viable option. The larger inhale dilutes the amount of acceptable CO2 collected in a larger sample volume, such that the minimum CO2 percentage may be reduced. Thus, in some embodiments, the minimum CO2 concentration for the breath sample to be considered valid may be reduced. For example, a breath sample may be considered valid if at least 2% CO2 is detected for a 2,500 mL volume or greater (e.g., up to about 3,500 mL volume), or at least 1.5% CO2 is detected for a 3,500 mL volume or greater. Thus, in some embodiments, an error alert is provided if the CO2 concentration drops below these threshold percentages as a function of collected sample volume.

The process then proceeds to step 208, which asks if a valid sample was collected? If not, the process provides an alert at step 209. The alert may be presented on a user interface, for example, on a display screen 315 of the analysis system 100. Additionally, or alternatively, the alert may be sent to a computer user-interface, e.g., via a mobile device application (e.g., iPhone). An alert may be sent to the patient 102, medical practitioner, and/or lab technician, such that a new breath may be obtained. Additionally, or alternatively, the alert may include a notification that the breath sample needs to be recollected.

A person of skill in the art will understands that in various embodiments, the process 208 may ask if an invalid sample was collected, such that the NO and YES process decisions after shown in FIG. 2 are switched at step 208.

Preferably, the alert is provided (e.g., displayed) within 1 minute to tell the medical professional that the breath collected is inadequate and needs to be retaken. Advantageously, this allows the patient 102 to provide another sample much earlier, rather than being called back at a much later time to retake the sample.

The process then advantageously proceeds to step 202 again, where a new breath sample is collected, and steps 204-208 are repeated. Advantageously, this process enables rapid determination of sample validity for breath diagnostics. Breath diagnostics, while non-invasive and convenient, face several sample validity limitations that can affect the accuracy and reliability of the results. A significant limitation is the appropriate collection of breath, which advantageously can be reliably determined in a rapid manner.

The typical process for VOC analysis requires a lab analysis for diagnostic purposes, which can be timely. Analyzing a faulty breath adds needless expense, and more importantly, takes away precious time from an early diagnosis. Typically, a faulty breath requires rescheduling sample collection with the patient 102, providing a new breath, and shipping out results, often weeks or months after initial sampling time. VOC analysis can be used to diagnose a number of diseases, include lung cancer, tuberculosis, and liver cirrhosis. It is highly advantageous to determine whether the collected diagnostic breath sample is valid at the time of collection, when a new sample can be obtained quickly from the patient 102. The process can be repeated until an appropriate sample is collected.

When a valid sample is collected, the process goes to step 210, which analyzes the breath sample to determine a medical/diagnostic outcome. It should be understood that analyzing the breath sample does not require the entirety of the breath sample, nor does it require the gaseous form of the breath sample. Indeed, analyzing the breath sample often includes looking at chemical components captured from the breath (e.g., the VOCs 106), some of which may have reacted with reagents. At step 210, a determination is made regarding a patient 102 parameter of interest.

In various embodiments, the collected breath sample has advantageously already passed through the microreactor 110, where target analytes (e.g., VOCs 106) are collected. When a valid sample is indicated, the microreactor 110 may be sent for analysis (e.g., by sending to a lab). Thus, the breath sample may be analyzed to determine a medical outcome. At step 212, an alert is provided that the breath sample is valid. Therefore, the breath sample or components thereof (e.g., the microreactor 110) may be sent out for analysis with confidence that the diagnostic results are based on a valid breath sample.

As described earlier, some steps may be skipped or may take place in a different order. As an example, in some embodiments step 210 may occur after step 212. For example, the analysis system 100 may first determine that the breath sample is valid, and then send the breath sample, or divert a portion thereof, through the microreactor 110 (or directly to a diagnostic analysis device). The process 200 then comes to an end.

Illustrative embodiments provide the analysis station 100 at the point of care (e.g., doctor office or testing place). Thus, shortly after the patient 102 provides the breath sample, the bag 302 containing the breath sample may be coupled to the analysis station 100 and determination of whether the breath is valid/adequate may begin right away.

Preferably, the sample is processing at a constant flow rate in the analyzer and the detection of faulty sample may occur while simultaneously processing the sample for VOC detection. In some embodiments, the microreactor may be removed and sent to an offsite location for mass spectrometry testing of particular VOC levels.

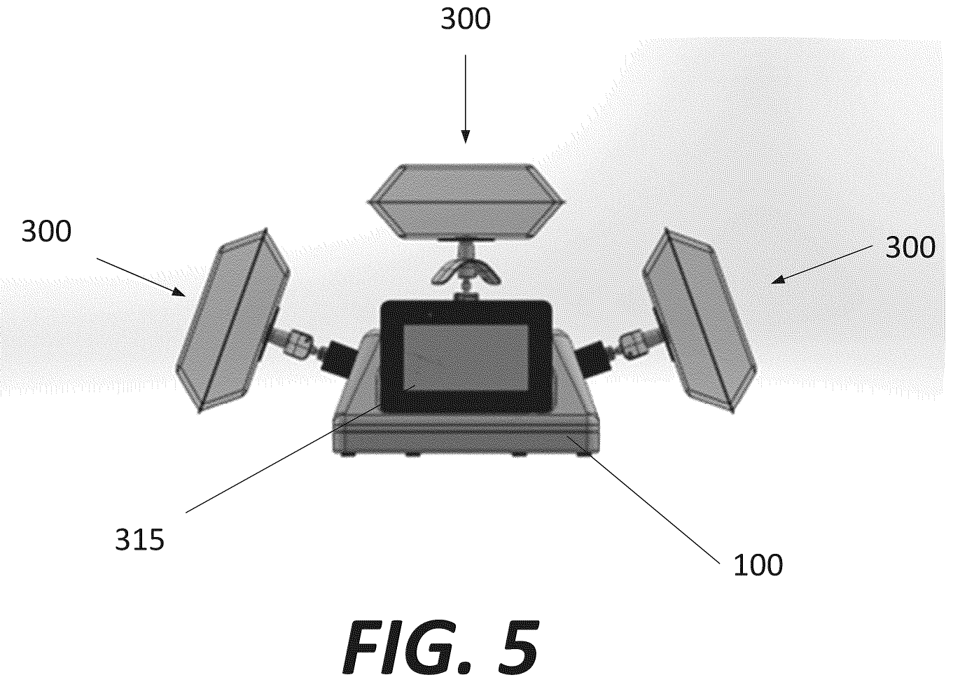

FIG. 5 schematically shows the analysis station 100 simultaneously coupled with three bags 302 for analysis. The evacuation station includes microfluidic channels (also referred to as pathways) that provide desirable fluid mechanics inside a pathway housing 250 within the station 100. The microfluidics chamber 250 is preferably designed to remove all tubing to reduce failure points. Additionally, the chamber 250 provides a closed system for each channel so that there is no cross-contamination of the breath samples. For example, some embodiments may provide 3 stations/chambers (as shown in the FIG. 5), but more can be added (e.g., manifold type design) as needed. Additionally, although discussion has been made with reference to the bags discussed herein, any fluid bag known in the art can be coupled with the analysis system 100, and illustrative embodiments are not limited to the collection device 300 disclosed herein. In various embodiments, the system 100 interface may be a luer lock interface, with a controllable flow rate and a customizable number of independent channels.

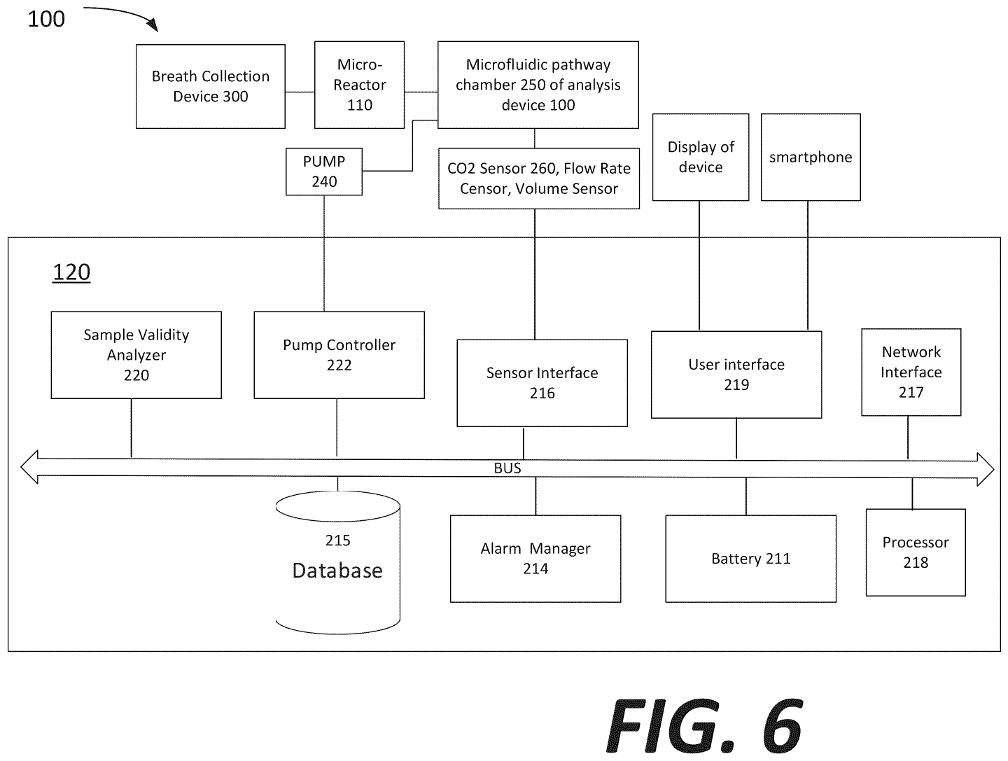

FIG. 6 schematically shows details of the fluid system controller 120 in accordance with illustrative embodiments of the invention. The controller 120 may be physically housed within the housing of the analysis system 100, for example, on the microfluidics controller PCBA 280 (Printed Circuit Board Assembly). Each of the components in FIG. 6 operatively connected by any conventional interconnect mechanism. FIG. 6 simply shows a bus communicating each the components. Those skilled in the art should understand that this generalized representation can be modified to include other conventional direct or indirect connections. Accordingly, discussion of a bus is not intended to limit various embodiments.

Indeed, it should be noted that FIG. 6 only schematically shows each of these components. Those skilled in the art should understand that each of these components can be implemented in a variety of conventional manners, such as by using hardware, software, or a combination of hardware and software, across one or more other functional components. For example, the pump controller 222 (discussed in detail below) may be implemented using a plurality of microprocessors executing firmware. As another example, the sample validity analyzer 220 may be implemented using one or more application specific integrated circuits (i.e., “ASICs”) and related software, or a combination of ASICs, discrete electronic components (e.g., integrated circuits), and microprocessors. Accordingly, the representation of the sample validity analyzer 220 and other components in a single box of FIG. 6 is for simplicity purposes only. In fact, in some embodiments, the sample validity analyzer of FIG. 6 is distributed across a plurality of different components—not necessarily within the same housing or chassis.

It should be reiterated that the representation of FIG. 6 is a significantly simplified representation of an actual analysis system controller 120. Those skilled in the art should understand that such a device has other physical and/or functional components, such as central processing units, other packet processing modules, and short-term memory. Accordingly, this discussion is not intended to suggest that FIG. 6 represents all of the elements of the fluid system controller 120. In fact, much of what was said here with regard to FIG. 6 can also be applied to components of the system 100 of FIG. 1.

As shown in FIG. 6, the evacuation station controller 120 can include a sensor interface 216 that interfaces with the sensors and the microfluidics chamber 250, a data storage 215, a network interface 217, a user interface 219, at least one battery 211, an alarm controller 214, a sample validity analyzer 220, a pump controller 222, and at least one processor 218.

As referenced previously, the microfluidics chamber 250 can be coupled to the cassette and/or the breath collection system 300 that was used to collect the breath from the patient 102. Many of the components of FIG. 6 directly or indirectly interact with and/or control the gas flowing through the microfluidics chamber 250.

The data storage 215 can include one or more of non-transitory computer readable media, such as flash memory, solid state memory, magnetic memory, optical memory, cache memory, combinations thereof, and others. The data storage 215 can be configured to store executable instructions and data used for operation of the controller 120. In certain implementations, the data storage can include executable instructions that, when executed, are configured to cause the processor 218 to perform one or more functions.

The data storage 215 may also include data relating to CO2% that relate to valid or invalid breath samples. In particular, the CO2% may be related to a particular patient 102 demographic, or total gas volume exhales by the patient 102. The sample validity analyzer 220 may communicate with the database to receive this information when making determinations regarding the validity of a breath sample. The validity analyzer 220 receives a CO2 signal from the CO2 sensor and compares this with the predetermined minimum CO2%. If the CO2% of the breath is equal to or greater than the minimum CO2%, then the breath is considered valid. If the CO2% of the breath is less than the minimum CO2%, then the breath is considered invalid. In some embodiments, the total volume of the sample may also be considered, and the minimum CO2% may be changed as a function of volume.

In some examples, the network interface 217 can facilitate the communication of information between the controller 120 and one or more other devices or entities over a communications network. For example, where the controller 120 is included in the evacuation station 100, the network interface 217 can be configured to communicate with a remote computing device such as a remote server or other similar computing device. The network interface 217 can include communications circuitry for transmitting data in accordance with a Bluetooth® wireless standard for exchanging such data over short distances to an intermediary device(s) (e.g., a base station, a “hotspot” device, a smartphone, a tablet, a portable computing device, and/or other devices in proximity of the wearable smart garment 110). The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a Wi-Fi™ communications link based on the IEEE 802.11 standard.

In certain implementations, the user interface 219 can include one or more physical interface devices such as input devices, output devices, and combination input/output devices and a software stack configured to drive operation of the devices. These user interface elements may render visual, audio, and/or tactile content. Thus the user interface 219 may receive input or provide output, thereby enabling a user to interact with the controller 120 (e.g., via a smartphone application or the display 315 on the evacuation system 100).

The controller 120 can also include at least one battery 211 configured to provide power to one or more components integrated in the controller 120. The battery 211 can include a rechargeable multi-cell battery pack. In one example implementation, the battery 211 can include lithium ion cells that provide electrical power to the other device components within the controller 120.

The sensor interface 216 can be coupled to one or more sensors (e.g., CO2 sensors) configured to monitor, quantify, and/or detect one or more parameters of the patient breath. As shown, the sensors may be coupled to the controller 120 via a wired or wireless connection. The sensors can include one or more CO2 sensors.

The sensor interface 216 can be coupled to any one or combination of sensors to receive other patient 102 data indicative of patient breath parameters. After data from the sensors has been received by the sensor interface 216, the data can be directed by the processor 218 to an appropriate component within the controller 120. For example, if CO2 data is collected by the CO2 sensor 224 and transmitted to the sensor interface 216, the sensor interface 216 can transmit the data to the processor 218 which, in turn, relays the data to a sample validity analyzer 220. The breath data can also be stored on the data storage 215.

In certain implementations, the alarm manager 214 can be configured to manage alarm profiles and notify one or more intended recipients of the validity or invalidity of a specified breath sample as determined by the sample validity analyzer 220. Alarm profiles may be created via the user interface 219 and stored in the data storage 215. The alarm profiles may indicate intended recipients. These intended recipients can include external entities such as users (patient 102, physicians, and monitoring personnel) as well as computer systems (monitoring systems, display devices, or emergency response systems). The alarm manager 214 can be implemented using hardware or a combination of hardware and software. For instance, in some examples, the alarm manager 214 can be implemented as a software component that is stored within the data storage 215 and executed by the processor 218. In this example, the instructions included in the alarm manager 214 can cause the processor 218 to configure alarm profiles and notify intended recipients using the alarm profiles. In other examples, alarm manager 214 can be an application-specific integrated circuit (ASIC) that is coupled to the processor 218 and configured to manage alarm profiles and notify intended recipients using alarms specified within the alarm profiles. Thus, examples of alarm manager 214 are not limited to a particular hardware or software implementation.

In some implementations, the processor 218 includes one or more processors (or one or more processor cores) that each are configured to perform a series of instructions that result in manipulated data and/or control the operation of the other components of the controller 120 and its various components. In some implementations, when executing a specific process (e.g., CO2 detection), the processor 218 can be configured to make specific logic-based determinations based on input data received, and be further configured to provide one or more outputs that can be used to control or otherwise inform subsequent processing to be carried out by the processor 218 and/or other processors or circuitry with which processor 218 is communicatively coupled. Thus, the processor 218 reacts to specific input stimulus in a specific way and generates a corresponding output based on that input stimulus. In some example cases, the processor 218 can proceed through a sequence of logical transitions in which various internal register states and/or other bit cell states internal or external to the processor 218 may be set to logic high or logic low.

As referred to herein, the processor 218 can be configured to execute a function where software is stored in a data store coupled to the processor 218, the software being configured to cause the processor 218 to proceed through a sequence of various logic decisions that result in the function being executed. The various components that are described herein as being executable by the processor 218 can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

A pump controller 222 can be coupled to one or more pumps 240 configured to control fluid flow of the breath sample from the breath collection device 300 through the microfluidic chambers 250. The pump controller 222 may receive a particular flow rate from the sample validity analyzer 220 or from the data storage 215. In some embodiments, the pump controller 222 may control a variety of different pump types. In various embodiments, the pump manipulates small volumes of fluids. The pumps 240 may advantageously produce precise and controlled fluid handling. The pumps 240 may include, among other things, peristaltic pumps, syringe pumps, and/or piezoelectric air pumps (e.g., a microblower by Murata Manufacturing Co., Ltd.).

The pump controller 222 can include, or be operably connected to, circuitry components that are configured to generate positive or negative pressure within the microfluids chamber 250 that may cause the gas to flow in a forward or reverse direction. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the pump 240 and under control of one or more processors (e.g., processor 218) to provide, for example, fluid flow from the breath collection device 300 at a desired flow rate and volume.

In some implementations, the processor 218 includes one or more processors (or one or more processor cores) that each are configured to perform a series of instructions that result in manipulated data and/or control the operation of the other components of the controller 120 and the system 100. In some implementations, when executing a specific process (e.g., CO2 detection, controlling the pump 240), the processor 218 can be configured to make specific logic-based determinations based on input data received, and be further configured to provide one or more outputs that can be used to control or otherwise inform subsequent processing to be carried out by the processor 218 and/or other processors or circuitry with which processor 218 is communicatively coupled. Thus, the processor 218 reacts to specific input stimulus in a specific way and generates a corresponding output based on that input stimulus. In some example cases, the processor 218 can proceed through a sequence of logical transitions in which various internal register states and/or other bit cell states internal or external to the processor 218 may be set to logic high or logic low.

As referred to herein, the processor 218 can be configured to execute a function where software is stored in a data store coupled to the processor 218, the software being configured to cause the processor 218 to proceed through a sequence of various logic decisions that result in the function being executed. The various components that are described herein as being executable by the processor 218 can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as preprogrammed hardware elements (e.g., application specific integrated circuits, FPGAs, programmable analog circuitry, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

As used in this specification and the claims, the singular forms “a,” “an,” and “the” refer to plural referents unless the context clearly dictates otherwise. Thus, in various embodiments, any reference to the singular includes a plurality, and any reference to more than one component can include the singular. For example, reference to “the pump” in the singular includes a plurality of pumps, and reference to “the bag” in the singular includes one or more bags and equivalents known to those skilled in the art. Thus, in various embodiments, any reference to the singular includes a plurality, and any reference to more than one component can include the singular.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein.

It is, therefore, to be understood that the foregoing embodiments are presented by way of example only and that, within the scope of the appended claims and equivalents thereto, inventive embodiments may be practiced otherwise than as specifically described and claimed. Illustrative embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Disclosed embodiments, or portions thereof, may be combined in ways not listed above and/or not explicitly claimed. Thus, one or more features from variously disclosed examples and embodiments may be combined in various ways. For example, it is to be understood that the present disclosure contemplates that, to the extent possible, one or more features of any embodiment can be combined with one or more features of any other embodiment.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the above discussion discloses various exemplary embodiments of the invention, it should be apparent that those skilled in the art can make various modifications that will achieve some of the advantages of the invention without departing from the true scope of the invention.

1-10. (canceled)

11. A method of analyzing a collected breath sample to determine if the breath is a faulty sample, the method comprising:
receiving a bag containing a collected patient breath sample;
fluidly coupling a bag with a cassette having reagents therein, and fluidly coupling the cassette with an analysis device having a fluidic pathway and a controller,
evacuating at least a portion of the patient breath sample from the bag, such that the patient breath sample passes through the cassette and reacts with the reagents therein to define a processed gas sample, at least a portion of the processed gas sample being received within the fluidic pathway of the analysis device;
analyzing the processed gas sample using a CO2 sensor of the analysis device to detect the concentration of CO2 in the processed gas sample; and
determining when a patient provided a faulty collected patient breath sample by comparing the CO2 concentration of the processed gas sample with a known faulty CO2 concentration.

12. The method as defined by claim 11, further comprising:
providing an alert that the collected patient breath sample was faulty.

13. The method as defined by claim 12, wherein the analysis device is configured to provide the alert within 1-minute of the processed gas sample being received within the fluidic pathway of the analysis device.

14. The method as defined by claim 11, wherein the analysis device comprises a controller configured to perform the analyzing and determining steps.

15. The method as defined by claim 11, wherein the processed gas sample is determined to be faulty if it contains less than about 2.5% CO2.

16. A method of analyzing a collected breath sample to determine if the breath is faulty, the method comprising:
receiving a collected patient breath sample in a breath collection device,
fluidly coupling the breath collection device with an analysis device having a fluidic pathway and a controller,
evacuating at least a portion of the patient breath sample from the breath collection device, such that at least a portion of the collected patient breath is received within the fluidic pathway of the analysis device;
analyzing the at least a portion of the collected patient breath sample using a CO2 sensor of the analysis device to detect the concentration of CO2 in the at least a portion of the collected patient breath; and
determining when a patient provided a faulty collected patient breath sample by comparing the CO2 concentration of the at least a portion of the collected patient breath sample with a known faulty CO2 concentration.

17. The method as defined by claim 16, further comprising:
providing an alert that the collected patient breath sample was faulty.

18. The method as defined by claim 17, wherein the analysis device is configured to provide the alert within 1-minute of the processed gas sample being received within the fluidic pathway of the analysis device.

19. (canceled)

20. The method as defined by claim 16, wherein the processed gas sample is determined to be faulty if it contains less than about 2.5% CO2.

21. The method as defined by claim 16, wherein the breath collection device is a bag having a normally closed valve.

22. The method as defined by claim 16, wherein the breath collection device is a bag having a normally closed valve with a teeth receiving area, the valve configured to open when a patient bites on the teeth receiving area.

23. The method as defined by claim 16, further comprising controlling a pump to control a fluid flow rate through the fluidic pathway of the analysis device and/or to extract gas from the breath collection device.

24. The method as defined by claim 16, further comprising:
receiving a second collected patient breath sample in a breath collection device after determining that the first collected patient breath sample was faulty;
fluidly coupling the breath collection device with an analysis device having fluidic pathway and a controller;
evacuating at least a portion of the second patient breath sample from the breath collection device, such that at least a portion of the second collected patient breath is received within the fluidic pathway of the analysis device;
analyzing the at least a portion of the second collected patient breath sample using a CO2 sensor of the analysis device to detect the concentration of CO2 in the at least a portion of the second collected patient breath; and
determining when a patient provided a faulty second collected patient breath sample by comparing the CO2 concentration of the at least a portion of the second collected patient breath sample with a known faulty CO2 concentration.

25. The method of claim 24, further comprising repeating the steps of collecting subsequent patient breath samples, evacuating at least a portion of the collected subsequent patient breath samples, analyzed the at least a portion of the collected subsequent patient breath samples, and determining when a patient provided a faulty subsequent patient breath samples, until a valid sample is obtained.

26. (canceled)

27. The method of claim 16, wherein the faulty sample is due to lack of alveolar breath or a leak in the channel.

28. A method of analyzing a collected breath sample to determine if the breath is valid, the method comprising:
receiving a bag having a collected patient breath sample therein,
fluidly coupling the bag with an analysis device having fluidic pathway and a controller,
evacuating at least a portion of the patient breath sample from the bag, such that at least a portion of the collected patient breath sample is received within the fluidic pathway of the analysis device;
analyzing the at least a portion of the collected patient breath sample using a CO2 sensor of the analysis device to detect the concentration of CO2 in the at least a portion of the collected patient breath sample; and
determining when a patient provided a valid collected patient breath sample by comparing the CO2 concentration of the at least a portion of the collected patient breath with a known valid CO2 concentration.

29. The method as defined by claim 28, further comprising:
providing an alert that the collected patient breath was not valid.

30. The method as defined by claim 28, wherein the analysis device is configured to provide the alert within 1-minute of the processed gas sample being received within the fluidic pathway of the analysis device.

31. The method as defined by claim 28, wherein the analysis device comprises a controller configured to perform the analyzing and determining steps.

32. The method as defined by claim 28, wherein the processed gas sample is determined to be valid if it contains more than about 2.5% CO2.

33-45. (canceled)
